(19) United States
(12) Patent Application Publication
(10) Pub. No.: US 2011/0014708 A1
(43) Pub. Date: Jan. 20, 2011

(54) NUCLEIC ACID FOR USE IN ALGAE AND USE THEREOF

(75) Inventors: Huai-Jen Tsai, Taipei City (TW); Si-Shen Li, Taipei City (TW)

Correspondence Address: WPAT, PC;INTELLECTUAL PROPERTY ATTORNEYS, 2030 MAIN STREET, SUITE 1300, IRVINE, CA, 92614, US

(73) Assignee: NATIONAL TAIWAN UNIVERSITY, Taipei City (TW)

(21) Appl. No.: 12/505,210

(22) Filed: Jul. 17, 2009

(51) Int. Cl. C12N 1/13; C07H 21/04; C12N 15/09

(52) U.S. Cl. 435/471; 536/23.5; 435/257.2

(57) ABSTRACT

The present invention provides a modified nucleic acid for expressing bovine lactoferricin (LFB) in algae, which consists of SEQ ID NO: 3. The present invention also provides a method of producing a foreign desired gene product in algae, comprising: (a) weakening or removing cell wall of the algae by a protein enzyme solution to make algae become suitable for gene transfer; (b) transferring a foreign gene encoding the desired gene product into the algae; and (c) expressing the foreign gene to produce the desired gene product. The present invention further provides a feed composition comprising a transgenic algae or its offspring, wherein the cell wall of said transgenic algae is weakened or removed by a protein enzyme solution.

### FIELD OF THE INVENTION

The present invention relates to a modified nucleic acid which is translated to bovine lactoferricin (LFB) in algae, and technology of transferring the nucleic acid and use there of.

### DESCRIPTION OF PRIOR ART

As a key cost-saving step, antibiotics are commonly employed in most fish aquacultures to prevent disease. However, the risk in this practice is that antibiotic-resistant pathogens may be selected and spread out along with the wastewater to cause serious environmental pollution. To address this problem, the present invention attempts to develop a safer, more effective and less expensive biological bactericide for aquaculture use.

Bovine lactoferricin (LFB) is a 3142-Da peptide derived from the N terminus of bovine lactoferrin (from Phe17 to Phe41). LFB can be generated by pepsin hydrolysis in the digestive tract (Bellamy W, Takase M, Yamauchi K, Wakabayashi H, Kawase K, Tomita M. Identification of the bactericidal domain of lactoferrin. Biochim Biophys Acta 1992;1121:130-6). LFB is an antimicrobial peptide that can kill or inactivate many pathogens, including Gram negative bacteria such as Escherichia coli, Klebsiella pneumoniae, Proteus vulgaris and Pseudomonas aeruginosa; Gram positive bacteria such as Clostridium paraputrificum, Corynebacterium ammoniagenes, Enterococcus faecalis, Listeria monocytogenes and Streptococcus bovis; parasites such as Eimeria stiedai, Giardia lamblia, and Toxoplasma gondii; fungi such as Aspergillus fumigatus; and viruses such as adenovirus, calicivirus and cytomegalovirus. Since LFB can suppress a wide variety of pathogens, it is an excellent antimicrobial peptide for aquaculture application. At the same time, LFB can only be produced by the hydrolysis of bovine lactoferrin under pepsin treatment in native source (Tomita M, Wakabayashi H, Yamauchi K, Teraguchi S, Hayasawa H. Bovine lactoferrin and lactoferricin derived from milk: production and applications. Biochem Cell Biol 2002;80:109-12). However, as described below, the present invention is able to bypass this limitation by creating conditions by which microalgae can generate LFB.

Microalgae have many advantages as bioreactors for the production of heterologous proteins. First, microalgae can produce complicated eukaryotic proteins after post-translational modification (Mayfield S P, Franklin S E, Lerner R A. Expression and assembly of a fully active antibody in algae. Proc Natl Acad Sci USA 2003;100:438-42). Second, although microalgae are eukaryotic, they can be cultured rapidly and economically. Third, microalgae are considered as a safe food because they are free from human pathogens and endotoxins.

Nannochloropsis oculata is a marine unicellular microalga which belongs to the class of Eustigmatophyceae and which has a spherical or slightly ovoid shape of about 2 to 4 mm in diameter. This microalga consists of a polysaccharide wall and contains only one chloroplast. Because N. oculata grows in a wide range of saline concentrations and contains a high amount of eicosapentaenoic acid, it is an important phytoplankton in the diet of fish larvae and an important organism for making ‘green water’ aquaculture ponds. In addition to the above advantages, N. oculata can be cultured in a closed system along the seashore and in the sea pond, thus eliminating the need for scarce freshwater and otherwise tillable land. Unlike yeast and cell line cultures, expensive medium and aseptic manipulation of N. oculata are not required. Moreover, as noted above, N. oculata can survive under extreme saline conditions; consequently, it can be grown in both freshwater and seawater aquaculture. Lastly, since the cryopreservation technique for N. oculata has been well developed, transgenic N. oculata is already available (Gwo J C, Chiu J Y, Chou C C, Cheng H Y. Cryopreservation of a marine microalga, Nannochloropsis oculata (Eustigmatophyceae). Cryobiology 2005;50:338-43).

### BRIEF DESCRIPTION OF THE DRAWINGS

FIG. 1 illustrates the expression vector, expression vector, phr-rLFB-Red, which enables microalgae to produce recombinant bovine lactoferricin (LFB) fused with RFP (DsRed). Hsp70A t RBCS 2: a promoter of heat shock protein 70A gene fused with a promoter of ribulose-1,5-bisphosphate carboxylase/oxygenase small subunit 2 gene of Chlamydomonas reinhardtii; rLFB: recombinant algae-codon-optimized LFB; DsRed: red fluorescent protein gene of Discosoma sp. (Clontech). Open arrows indicate the sites where digestion by pepsin releases the functional domain LFB; Nhel and EcoRV are cutting sites for inserting the DNA fragment which encodes the fusion protein (LFB and DsRed) into the pCB740 vector. DF and DR are the primers used for screening of microalgal transformants harboring phr-rLFB-Red. A 450-bp PCR product is expected to amplify.

FIG. 2 shows Nannochloropsis oculata cells lysed by synthetic gastric juice treatment. N. oculata cells were cultured at log phase and treated by synthetic gastric juice under gentle shaking at 37° C. in the dark for certain times as indicated. The number of intact algal cells decreased when the time of synthetic gastric acid treatment increased.

FIG. 3 shows PCR detection of the transformed microalgae. Microalgae were electroporated with 10 μgml⁻¹ linear form plasmid (phr-rLFB-Red) under various combinations of pepsindigestion duration and voltages as indicated. After the third generation, 1 ml microalgae (1×10⁷ cells) were treated with 50 μg DNase I at 37° C. for 1 h and inactivated at 65° C. for 20 min before their genomic DNA was analyzed by PCR. Lane M: molecular marker; lane N: negative control (no DNA template); lane P: positive control (1 ng of linear plasmid: phrrLFB-Red), lanes 1-11: single colony of microalgae; laneW: wild-type alga that was incubated with 10 μgml⁻¹ linear plasmid, but without electroporation. When DF and DR primers were used, a 450-bp PCR product was expected to be produced by transgenic microalgae. Amplification of 18S rRNA served as an internal control.

FIG. 4 shows stable lines of transgenic algal clones. After transgenic microalgae were cultured on an agar plate for 60 days, two transformants (indicated as 0829A and 0829B) showed a predominant red around the colonies (A and D). Red fluorescent signal was observed under fluorescent microscope equipped with a 560 nm excitation filter and a 610 nm emission filter (E), whereas the wild type (B) and unstable transformants (A) neither showed red around the colonies nor appeared with red fluorescent signal under fluorescence microscopy (C).

FIG. 5 shows RT-PCR analysis of microalgae. (A) Transgene and the primers designed for detecting the transgene (PF and LR) and its mRNA (LF and LR). Primers LF and LR were used to amplify a 68-bp DNA fragment from the transcript of recombinant bovine lactoferricin (rLFB) cDNA, whereas primers PF and LR were used to amplify a 380-bp PCR fragment from the contaminated plasmid DNA. (B) RT-PCR analysis of mRNA extracted from 20 ml microalgae (1×10⁷ cells ml⁻¹) after treatment at 42° C. for 16 h. Lane M: molecular marker; lane P: positive control (1 ng of phr-rLFB-Red); lane N: negative control (no DNA template); lane W: wild-type microalga, lanes 1-4: transgenic microalgae, while 18S rRNA served as an internal control.

FIG. 6 shows protein and western blot analyses of microalgae. After 50 ml of wild type (W) and transgenic (T) microalgae (1×10⁷ cells ml⁻¹) were treated at 42° C. for 16 h, their total proteins were extracted and analyzed by SDS-polyacrylamide gel electrophoresis on a 12% gel (A). The gel was transferred into nitrocellulose membrane and reacted with monoclonal antiserum against DsRed (B). Arrow indicates the tetramer of recombinant fusion protein LFB-DsRed produced by transgenic microalga.

FIG. 7 shows quantitative analysis of proteins produced by transgenic microalga. After the transgenic microalga 0829A was treated at 42° C. for 16 h, the total proteins were extracted and analyzed by SDS-PAGE (upper panel). The protein profile was then analyzed by the TotalLab computer program (lower panel). Arrows indicate the recombinant protein LFB-DsRed band and peak produced by transgenic microalga. This exogenous protein was 4.27% of total soluble proteins.

FIG. 8 shows bactericidal potency on the lysates extracted from transgenic microalgae. The transgenic alga 0829A was cultured at log phase, induced by heat shock; then the cells were harvested and resuspended in gastric acid for 4 h. Cell lysate was obtained, spotted on the disc (0.5 cm in diameter) and placed on the agar plate containing Vibrio parahaemolyticus. Inhibition zone was evaluated when lysates were extracted from different volumes (as indicated) of microalgae. Different dosages of Ampicillin were used as a positive control for calculating the potency of functional domain of LFB produced by transgenic microalgae after gastric juice treatment.

FIG. 9 shows that mortality of Penaeus monodon was related to the number of days after WSSV injection in the oral vaccine infection test. Time post infection: the number of days after injection. Cumulative mortality (%): cumulative mortality of Penaeus monodon. The star sign marked the group which had significant difference from the positive control. The Challenge dose is equal to LD90. Positive and negative control: general feed. Transgenic algae: feed comprising VP28 transgenic algae. Half wild-type algae plus half transgenic algae: feed comprising half wild-type algae plus half VP28 transgenic algae. Wild-type: feed comprising wild-type algae.

### SUMMARY OF THE INVENTION

The present invention relates to a modified nucleic acid for expressing in algae, which consists of nucleotides of SEQ ID NO: 3, wherein the nucleotides can be translated to bovine lactoferricin (LFB) in algae. The present invention also relates to a method of producing a foreign desired gene product in algae, comprising: (a) weakening or removing cell wall of the algae by a protein enzyme solution to make algae become suitable for gene transfer; (b) transferring a foreign gene encoding the desired gene product into the algae; and (c) expressing the foreign gene to produce the desired gene product. The present invention further relates to a feed composition comprising a transgenic algae or its offspring, wherein the cell wall of said transgenic algae is weakened or removed by a protein enzyme solution.

### DETAILED DESCRIPTION OF THE INVENTION

The present invention transferred three different foreign genes into Nannochloropsis oculata respectively, comprising a gene encoding a fusion protein of LFB and red fluorescent protein (LFB-DsRed) for replacing antibiotics, a gene encoding an envelop protein VP28 of white spot syndrome virus for being used as vaccine, or a gene encoding growth factor rYGH in Acanthopagrus latus for promoting the growth of fish. The examination of DNA, RNA, protein and biological activity test clearly demonstrate that the present invention successfully transferred the foreign genes into N. oculata and expressed three foreign proteins with different sources, characteristics and functions. These foreign proteins expressed in N. oculata have biological activity.

Therefore, the present invention provides a modified nucleic acid for expressing bovine lactoferricin (LFB) in algae, which consists of SEQ ID NO: 3. In a preferred embodiment, the nucleic acid is further operably linked to a sequence encoding fluorescent protein and is translated to a fusion protein of LFB and fluorescent protein, wherein the fluorescent protein is a red fluorescent protein (DsRed), providing a sequence consisting of SEQ ID NO: 2 which is translated to a fusion protein of LFB and red fluorescent protein (LFB-DsRed). The fluorescent protein is a fluorescent marker for screening transgenic algae, and a pepsin cutting site is located in the junction between LFB and DsRed. In a more preferred embodiment, the nucleic acid linked to a sequence encoding fluorescent protein is further operably linked to a promoter, wherein the promoter consists of SEQ ID NO: 1.

In a preferred embodiment, the algae is microalgae, wherein the microalgae is selected from the group consists of Chlorella from sea and fresh water, Chlamydomonas, Volvox, Cheatoceros, Phaeodactylu, Skeletonema, Navicul, Nitzschia, Thalassiosira, Amphora, Nannochloris, Nannochloropsi, Tetraselmis, Dunaliella, Spirulina, Microcysti, Oscillatoria, Isochrysis, Paviova and Dinophyceae. In a more preferred embodiment, the algae is Nannochloropsis oculata.

The present invention also provides a method of producing a foreign desired gene product in algae, comprising: (a) weakening or removing cell wall of the algae by a protein enzyme solution to make algae become suitable for gene transfer; (b) transferring a foreign gene encoding the desired gene product into the algae; and (c) expressing the foreign gene to produce the desired gene product.

The term “foreign gene” used herein means a gene which is not originated from treated algae. The foreign gene can be originated from bacteria, fungi, virus, animals or plants. In a preferred embodiment of the present invention, the foreign gene comprises a gene encoding a fusion protein of LFB and red fluorescent protein (LFB-DsRed), a gene encoding an envelop protein VP28 of white spot syndrome virus, or a gene encoding growth factor rYGH of Acanthopagrus latus.

The term “protein enzyme” used herein means an enzyme which can break down proteins into smaller ones. There are no limitations on the origin of the protein enzyme of the invention and/or for the use according to the invention. Thus, the term protein enzyme includes not only natural or wild-type protein enzymes, but also any mutants, variants, fragments etc. thereof exhibiting protein enzyme activity, as well as synthetic protein enzymes, such as shuffled protein enzymes, and consensus protein enzymes.

The “protein enzyme” used in the present invention includes but not limited to pepsin A. The term “protein enzyme solution” used herein means a mixed solution containing a protein enzyme, wherein the solution provides an environment in which said enzyme is activated. In a preferred embodiment, the protein enzyme solution is synthetic gastric juice comprising hydrochloric acid, potassium chloride, and pepsin A. In a more preferred embodiment, the synthetic gastric juice comprises 150 mM hydrochloric acid, 15 mM potassium chloride, and 5% pepsin A.

In the traditional electroporation transgenic method, yeast cell wall is treated by cellulose for electroporation, but the cellulose is ineffective for algae cell wall. The protein enzyme used in the present invention can efficiently weaken or remove algae cell wall and make the algae become suitable for electroporation.

In a preferred embodiment, the transferring of the foreign gene encoding the desired gene product into the algae is accomplished by electroporation. The desired gene product expressed in algae has biological activity comprising antibacterial activity, antiviral activity, or increasing growth rate. In a more preferred embodiment, the biological activity comprises antibacterial activity for Vibrio parahaemolyticus, antiviral activity for white spot syndrome virus, or increasing growth rate 2 to 3 times.

In a preferred embodiment, the algae is microalgae, wherein the microalgae is selected from the group consists of Chlorella from sea and fresh water, Chlamydomonas, Volvox, Cheatoceros, Phaeodactylu, Skeletonema, Navicul, Nitzschia, Thalassiosira, Amphora, Nannochloris, Nannochloropsi, Tetraselmis, Dunaliella, Spirulina, Microcysti, Oscillatoria, Isochrysis, Paviova and Dinophyceae. In a more preferred embodiment, the algae is Nannochloropsis oculata.

The present invention also provides a transgenic algae with cell wall weakened or removed by a protein enzyme solution, wherein the transgenic algae comprises a transferred foreign gene encoding a foreign gene product, wherein the foreign gene is originated from bacteria, fungi, virus, animals or plants.

In a preferred embodiment, the foreign gene comprises a gene encoding a fusion protein of LFB and red fluorescent protein (LFB-DsRed), a gene encoding an envelop protein VP28 of white spot syndrome virus, or a gene encoding growth factor rYGH of Acanthopagrus latus. The foreign gene product is expressed in algae and has biological activity comprising antibacterial activity, antiviral activity, or increasing growth rate. In a more preferred embodiment, the biological activity comprises antibacterial activity for Vibrio parahaemolyticus, antiviral activity for white spot syndrome virus, or increasing growth rate 2 to 3 times.

In a preferred embodiment, the protein enzyme solution is synthetic gastric juice comprising hydrochloric acid, potassium chloride, and pepsin A. In a more preferred embodiment, the synthetic gastric juice comprises 150 mM hydrochloric acid, 15 mM potassium chloride, and 5% pepsin A.

In a preferred embodiment, the algae is microalgae, wherein the microalgae is selected from the group consists of Chlorella from sea and fresh water, Chlamydomonas, Volvox, Cheatoceros, Phaeodactylu, Skeletonema, Navicul, Nitzschia, Thalassiosira, Amphora, Nannochloris, Nannochloropsi, Tetraselmis, Dunaliella, Spirulina, Microcysti, Oscillatoria, Isochrysis, Paviova and Dinophyceae. In a more preferred embodiment, the algae is Nannochloropsis oculata.

The present invention further provides a feed composition comprising a transgenic algae or its offspring, wherein the cell wall of said transgenic algae is weakened or removed by a protein enzyme solution. The transgenic algae comprises a transferred foreign gene encoding a foreign gene product, wherein the foreign gene is originated from bacteria, fungi, virus, animals or plants.

In a preferred embodiment, the foreign gene comprises a gene encoding a fusion protein of LFB and red fluorescent protein (LFB-DsRed), a gene encoding an envelop protein VP28 of white spot syndrome virus, or a gene encoding growth factor rYGH of Acanthopagrus latus. The foreign gene product is expressed in algae and has biological activity comprising antibacterial activity, antiviral activity, or increasing growth rate. In a more preferred embodiment, the biological activity comprises antibacterial activity for Vibrio parahaemolyticus, antiviral activity for white spot syndrome virus, or increasing growth rate 2 to 3 times.

In a preferred embodiment, the algae is microalgae, wherein the microalgae is selected from the group consists of Chlorella from sea and fresh water, Chlamydomonas, Volvox, Cheatoceros, Phaeodactylu, Skeletonema, Navicul, Nitzschia, Thalassiosira, Amphora, Nannochloris, Nannochloropsi, Tetraselmis, Dunaliella, Spirulina, Microcysti, Oscillatoria, Isochrysis, Paviova and Dinophyceae. In a more preferred embodiment, the algae is Nannochloropsis oculata.

In a preferred embodiment, the composition is used to feed an aquatic organism. In a more preferred embodiment, the aquatic organism is fish or shrimp.

### EXAMPLES

The examples below are non-limiting and are merely representative of various aspects and features of the present invention.

### Example 1

### Materials and Methods

### Plasmid Construction and Preparation

An inducible promoter of algae expression vector, Hsp70A plus RBCS 2 (SEQ ID NO: 1), was obtained from Schroda et al. (Schroda M, Blocker D, Beck C F. The HSP70A promoter as a tool for the improved expression of transgenes in Chlamydomonas. Plant J 2000;21:121-31). A cDNA of fusion protein (SEQ ID NO: 2), an algae-codon-optimized LFB (SEQ ID NO: 3), which was fused with a DsRed reporter and driven by this inducible promoter, was generated by the following sequential PCR. Plasmid pDsRed 2-1 (Clontech) containing the DsRed cDNA of Discosoma sp. was used as a template. Three forward primers and one reverse primer were designed: CF3 (GCTAGCACCGGTCGCCACCATGTTCAAATGTCGTCGTTGGCAATGGCGT) (SEQ ID NO: 4), containing an Nhel cutting site and a 19-bp Kozak sequence; CF2 (GCAATGGCGTATGAAAAAATTAGGTGCTCCTTCTATTAC) (SEQ ID NO: 5), containing a 78-bp algae-codon-optimized LFB cDNA and two pepsin-digestion sites, and CF1 (CTTCTATTACATGTGTACGTCGTGCTTTCATGGCCTCCT) (SEQ ID NO: 6), containing the partial cDNA of N terminus (10 bp) of DsRed; one reverse primer, CR (ATTTGTGATGCTATTGCTTTATTTGTAACCATT) (SEQ ID NO: 7), containing partial cDNA of C terminus (10 bp) of DsRed and an EcoRV cutting site. Primers CF1 and CR were used in the first PCR reaction under the following conditions: 5 cycles of denaturation at 94° C. for 30 s, annealing at 30° C. for 15 s and extension at 72° C. for 90 s, followed by 20 cycles of denaturation at 94° C. for 30 s, annealing at 60° C. for 15 s and extension at 72° C. for 90 s. The resultant 734-bp PCR product was extracted from a 2% agarose gel after electrophoresis. This PCR product was used as the template DNA for the second PCR reaction using primers CF2 and CR. The resultant 763-bp PCR product was used as the template for the third PCR reaction by using primers CF3 and CR. The final 802-bp PCR product was cloned into plasmid pGEM-T easy. After the resultant plasmid was digested with Nhel and EcoRV, it was inserted into the NheI-EcoRV cut of pCB740 to generate the algal LFB expression plasmid, phr-rLFB-Red. This plasmid was linearized by SacII and readied for gene transfer use.

### Culture Conditions and Synthetic Gastric Juice Treatment of Microalgae

N. oculata cells obtained from Taiwan Fisheries Research Institute were cultured in f\2 medium at 28° C. with an illumination of white fluorescent tubes (Taiwan Fluorescence Company: FL40D\38) on a 12:12 h light\day cycle. For preparing protoplasts, 5 ml of N. oculata cells (1×10⁷ cells ml⁻¹) were collected by centrifugation at 8000×g for 10 min at 4° C. The pellet was washed twice with 1 ml of sterilized seawater, resuspended in 500 μl of synthetic gastric juice, and incubated at 37° C. for different times in the dark with gentle shaking. After treatment, cells were washed twice with 1 ml of sterilized seawater to terminate enzymatic activity and observed by microscope. Finally, the pellet was resuspended in 0.2 ml electroporation buffer and chilled on ice for 10 min to prepare for electroporation.

### Gene Transfer by Electroporation

One half (0.1 ml) of N. oculata protoplasts resuspended in the electroporation buffer was respectively added to 10 μg of 3 kinds of linearized foreign gene fragments: gene encoding a fusion protein of LFB and red fluorescent protein (LFB-DsRed), gene encoding an envelop protein VP28 of white spot syndrome virus, or gene encoding growth factor rYGH of Acanthopagrus latus. Then, the gene transfer was performed by electroporator (ECM 2001, BTX, USA) under various voltages ranging from 1 to 2 kV at 20 μs pulse time for 10 pulses to generate the transgenic N. oculata.

### Regeneration and Growth

After electroporation, N. oculata cells were transferred to a glass tube containing 1 ml of fresh f\2 medium and cultured at 28° C. for 24 h with agitation at 200 rpm. Twenty microlitres of algal cells grown at log phase (1×10⁷ cells ml⁻¹) were spread on an agar plate containing f\2 medium and incubated at 28° C. for 5 to 7 days. Colonies were picked up and cultured in liquid medium as described above for subsequent analysis.

### Genomic DNA Extraction

The genomic DNA extraction of N. oculata followed the description of Dawson et al. (Dawson H N, Burlingame R, Cannons A C. Stable transformation of Chlorella: rescue of nitrate reductase-deficient mutants with the nitrate reductase gene. Curr Microbiol 1997;35:356-62) with some modifications. Microalgal cells were harvested from 5 ml (approximately 1×10⁷ cells ml⁻¹) of culture medium and resuspended in 500 μl of buffer solution (54 mM hexadecyltrimethylammonium bromide, 0.25 mM Tris (pH 8.0), 1.4 M NaCl, 10 mM EDTA, and 2% β-mercaptoethanol). The mixture was incubated at 65° C. for 2 h and shaken every 15 min. After incubation, an equal volume of phenol-chloroform was added, and the aqueous phase was recovered after 5 min of centrifugation at 8000×g at 25° C. for 10 min. We extracted several times until the aqueous layer was no longer cloudy. The genomic DNA was precipitated with two volumes of 100% ethanol, centrifuged at 8000×g for 15 min, washed with 70% ethanol, dried, and resuspended in 30 μl of TE buffer.

Screening the Putative Transgenic N. oculata by PCR Analysis

Two oligonucleotide primers were synthesized for detection of the existence of the transferred DNA fragment, DsRed cDNA, by PCR analysis: a forward primer (DF: CCTCCTCCGAGAACGTCATCACCGAG) (SEQ ID NO: 10) and a reverse primer (DR: CCTCGGTGCGCTCGTACTGCT) (SEQ ID NO: 11). The primers for detection of the endogenous 18S rRNA gene, which served as an internal control, were forward primer (GCGGAGGAAAAGAACTAACCAGGATT) (SEQ ID NO: 12) and reverse primer (AACGCCATGGCACACCGC) (SEQ ID NO: 13). Each PCR sample consisted of 20 μl of solution containing 10 to 20 ng of template, 10 pmol of each primer, 25 μM of each dNTP, and 5 units of Taq enzyme (Viotech, Taiwan) in a 10× PCR buffer. Amplification was performed with a Perkin-Elmer Cetus DNA Thermal Cycler (USA). PCR consisted of 25 cycles of denaturation at 94° C. for 1 min, annealing at 55° C. for 1 min, and extension at 72° C. for 1 min, followed by 10 min extension at 72° C. PCR products (10 μl) were subjected to electrophoresis on a 3% NuSieve GTG agarose gel (FMC BioProducts, USA).

Screening the Putative Transgenic N. oculata by Fluorescent Microscope

In addition to using PCR to screen the putative transgenic clones of microalgae, we also used fluorescent microscopy to detect the transgenic clones because the transgene contains DsRed. After electroporation, we cultured microalgae on agar plates for two months and observed the expression of red fluorescence at 610 nm under a stereo dissecting microscope (MZ12, Leica) equipped with a fluorescent module having a DsRed filter cube (Kramer Scientific Corp., Valley Cottage, N.Y., USA).

Preparation of mRNA

After 40 ml of wild type and transgenic microalgae (1×10⁷ cells ml⁻¹) were treated at 42° C. for 16 h, microalgal cells were harvested from culture medium and resuspended in 1 ml of REzol reagent (PeproTech, Rocky Hill, N.J., USA). The mixture was shaken for 30 s and incubated at 25° C. for 5 min. After incubation, 200 μl of phenol-chloroform were added, and the aqueous phase was recovered after 5 min of centrifugation at 8000×g at 4° C. for 10 min and the DNA was digested by DNase at 37° C. for 1 h. The mRNA was precipitated with 0.6 volume of isopropanol, incubated for 2 h, centrifuged at 8000×g for 15 min at 4° C., and, finally, washed with 70% ethanol, dried, and resuspended in 30 μl of DEPC water.

### Reverse Transcription-Polymerase Chain Reaction (RT-PCR)

First-strand cDNA was synthesized using the SuperScript II Preamplification System (Gibco BRL). Primers LF (GTCGTTGGCAATGGCGTATGA) (SEQ ID NO: 14) and LR (GCCATGAAAGCACGTACACATGTAAT) (SEQ ID NO: 15) were used to amplify an expected 68-bp PCR product from the transcript of LFB cDNA. Primers PF (CGCTGAGGCTTGACATGATTGGTG) (SEQ ID NO: 16) and LR were used to amplify a 380-bp PCR fragment from the contaminated plasmid DNA which is not eliminated by DNase. If the 380-bp PCR fragment did not appear, the results of algal transcriptional experiments were authentic.

### Induction, Protein Extraction and Western Blot Analysis

Fifty millilitres (about 1×10⁷ cells ml⁻¹) of the transformed N. oculata were treated by shifting to 42° C. for 16 h to express the exogenous protein driven by an inducible promoter of Hsp70A and RBCS 2. After induction, the total proteins were extracted from the transgenic N. oculata using the protocol of Hawkins and Nakamura (Hawkins R L, Nakamura M. Expression of human growth hormone by the eukaryotic alga, Chlorella. Curr Microbiol 1999;38:335-41) with a modification. Briefly, after microalgae were collected by centrifuging for 10 min at 8000×g at 4° C., the pellet was resuspended in 500 μl of extraction buffer. The cell suspension was transferred to a microtube (Axygen Scientific, USA) containing 0.5 g of glass beads (Sigma, USA) and broken by a mini-bead beater (BioSpec Products, Bartlesville, OK, USA) at room temperature for 30 s with a 90 s interval on ice, a process repeated for 15 times. The supernatant was centrifuged for 15 min at 10,000×g at 4° C., and 50 ml of sample loading buffer (1 mM EDTA, 250 mM Tris-HCl (pH 6.8), 4% SDS, 2% β-mercaptoethanol, 0.2% bromophenol blue, 50% glycerol) was added to dissolve the pellet. Prior to SDS-PAGE analysis, samples were boiled for 40 min and then centrifuged for 5 min at 10,000×g. The supernatant was electrophoresed on a 12% SDSpolyacrylamide gel. A mouse monoclonal antibody against DsRed (Clontech, USA) was used to detect the recombinant fusion protein of LFB-DsRed. The final dilution of monoclonal antibody was 1:2000, and an alkaline phosphatase-conjugated anti-rabbit IgG (Santa Cruz, USA) was used as the secondary antibody. The procedures for western blot analysis were described previously (Tsai H J, Lin K L, Chen T T. Molecular cloning and expression of yellowfin porgy (Acanthopagrus latus Houttuyn) growth hormone cDNA. Comp Biochem Physiol 1993;104B:803-10).

Bactericidal Activity of Recombinant LFB Produced by Transgenic N. oculata

To detect the efficacy of bactericidal activity of recombinant LFB produced by the transgenic microalgae cloned in the present invention, a filter paper disc assay was performed. Different volumes of microalgae were treated by synthetic gastric juice for 4 h, spotted on a paper disc (0.5 cm at diameter), placed on a covered agar plate containing V. parahaemolyticus and cultured at 37° C. for 16 h. This in vitro inhibition zone assay was performed three times. For in vivo bioassay, the small model freshwater fish, Japanese medaka (Oryzias latipes), which had been adapted from freshwater to seawater seven days prior, was used. Then, either wild type or transgenic algae was delivered into the stomach of medaka in different amounts (1×10⁶ or 1×10⁸ cells per fish) by tomcat catheter tube through the mouth. After oral-in-tube delivery of algae for 6 h, both wild type and transgenic medaka were infected with V. parahaemolyticus, again using oral-in-tube delivery. The survival rate of microalgae-treated medaka was calculated 24 h after V. parahaemolyticus transfection. This in vivo assay was performed by two independent trials.

### Example 2

### Plasmid Construction

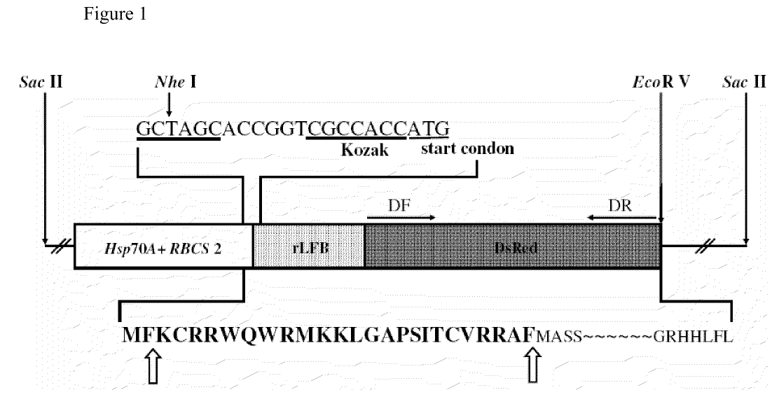

Four primers (CF1, CF2, CF3 and CR) were used to obtain various various PCR products from pDsRed 2-1 (Clontech) as a template. The nucleic acid sequence of each PCR product was confirmed by DNA sequencing. The final plasmid of phr-rLFB-DsRed with a molecular mass of 4.4 kb was constructed with a Kozak consensus sequence consisting of a 78-bp algae-codon-optimized LFB peptide, including two pepsin-digestion sites at Phe2-Lys and Phe26-Met and a DsRed protein (FIG. 1). This fusion protein was driven by a promoter of HSP70A combined with RBCS 2 from Chlamydomonas reinhardtii.

### Example 3

### Preparation of the Protoplasts of N. oculata for Gene Transfer

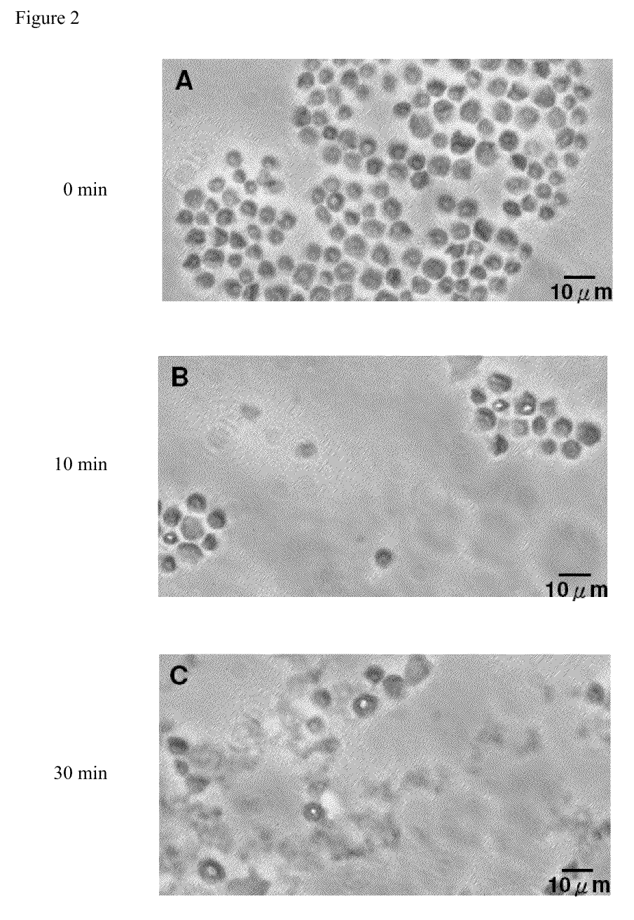

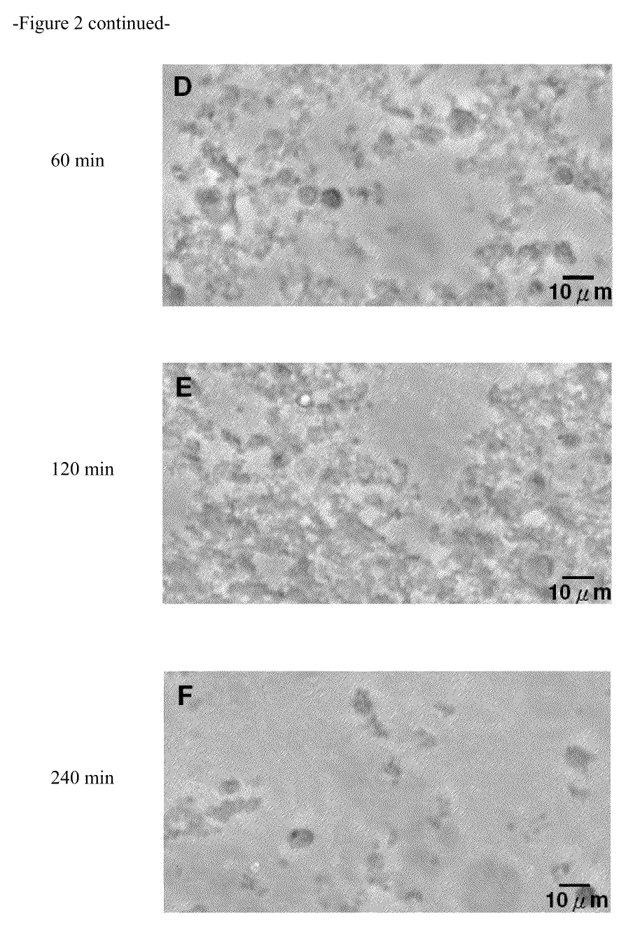

After N. oculata cells were cultured at log phase to a density of 1×10⁷ cells, they were treated with synthetic gastric juice and gentle shaking at 37° C. for 0, 30, 60, 120 and 240 min in the dark. The number of intact algae cells decreased as the time of synthetic gastric acid treatment increased (FIG. 2). Thus, when synthetic gastric acid was treated for 0, 10, 30, 60, 120 and 240 min, a total of 147, 20, 8, 4, 0 and 0 algal cells remained intact, respectively. Algal protoplasts were took after treating for 30 min with synthetic gastric juice and electroporated in the presence of SacII-cut phrrLFB-DsRed for 20 ms pulse time and 10 pulses, either at 1 or at 2 kV. There were 1248 and 147 clones to generate the transgenic N. oculata, respectively. Protoplasts were also prepared from the algae that were treated with gastric juice for 60 min; 24 and 21 algal clones survived after they were electroporated in the presence of SacII-cut phr-rLFB-DsRed for 20 ms pulse time and 10 pulses, either at 1 or 2 kV, respectively.

### Example 4

### Screening the Putative Transgenic Clones of N. oculata by PCR

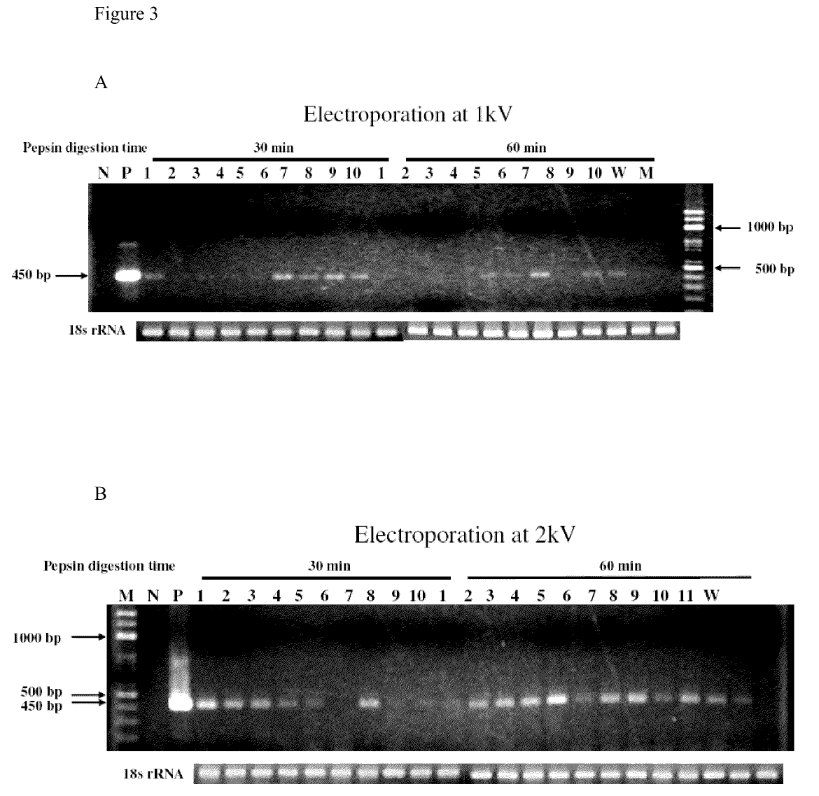

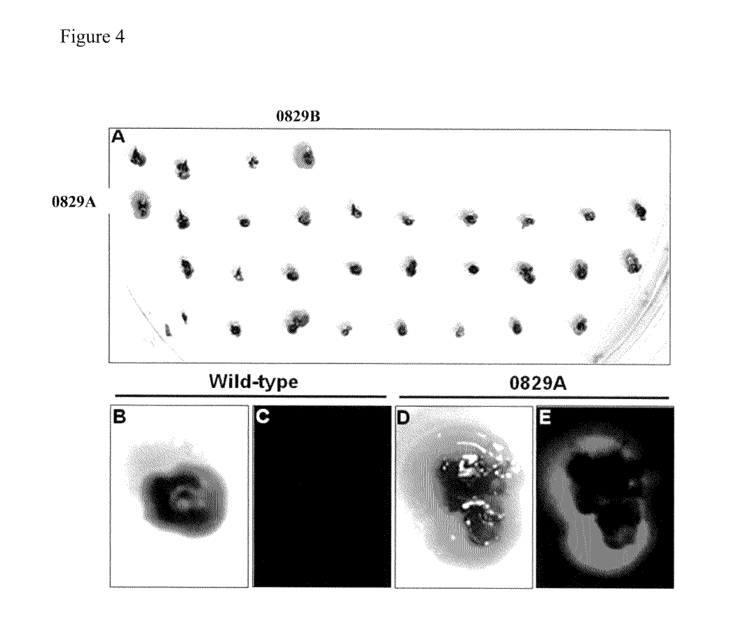

Since antibiotics did not affect the growth of microalgae in solid plate culture, N. oculata cells were observed to grow normally in the medium, whether or not the cells contained the plasmid phr-rLFBRed. Thus, in order to identify if each colony contained the transferred gene after electroporation and culturing at the third generation, we used PCR. The primers DF and DR were used to carry out the PCR amplification for screening of microalgae harboring phr-rLFB-Red (FIG. 1). In total, 491 transformants grown on the culture plates were screened. These transformants were randomly selected from the group which had been electroporated at 1 kV and treated with gastric juice for either 30 or 60min. As expected, a single 450-bp PCR product was generated for the samples from either the 30min group (FIG. 3A, left lanes 1, 6-9) or the 60 min group (FIG. 3A, right lanes 4-6, 8 and 9) when the primers DF and DR were used. The molecular mass of this positive band was similar to that of the PCR product amplified from the DNA fragment, a SacII-cut phr-rLFB-DsRed DNA, which was used for gene transfer. The significance of the similarity lies in the fact that the foreign DNA fragments had been transferred into microalgae. However, although more than 50% of examined clones harbored the transferred DNA fragment from the microalgae electroporated at 1 kV, almost every clone harbored the transferred gene from the microalgae electroporated at 2 kV (FIG. 3B). At the same time, however, most of these transformants completely lost their exogenous DNA fragment when they were examined after culturing for 15 months, suggesting that the transgene is not stably inherited in most N. oculata after a long period of culture. After intensive screening, only two PCR-positive clones were found after they have been cultured for 22 months, and these were designated as stable lines 0829A and 0829B. In spite of low possibility of obtaining a stable line, one still has a chance to discover stable transgenic strain after massive screening. Interestingly, when the transgenic microalgae grew on agar plates more than 60 days, fluorescence microscopy detected a predominant red fluorescent signal, but only around the colonies of stable lines 0829A and 0829B (FIGS. 4A, D and E). Both signals were so strong that 0829A and 0829B could be observed by viable light under photomicroscope (FIG. 4A), but since the signal of 0829A was stronger than 0829B, stable line 0829A was chosen for further experiments.

### Example 5

### Using Reverse Transcription-Polymerase Chain Reaction

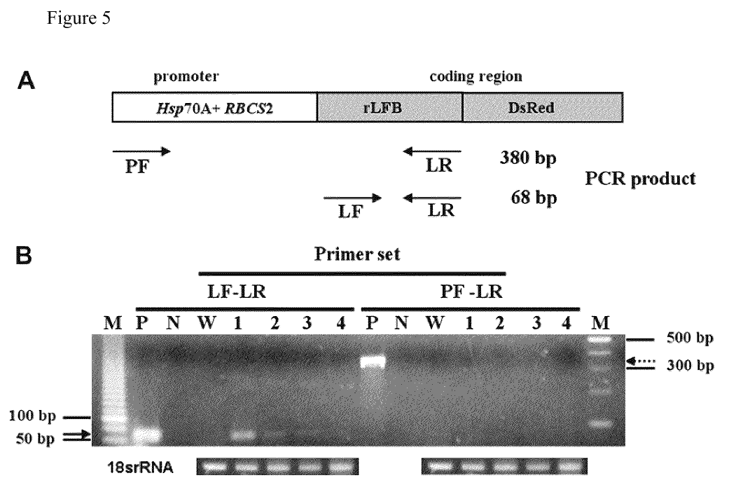

RT-PCR was used to analyze the RNAs extracted from 20 ml PCRpositive microalgae (1×10⁷ cells ml⁻¹) at third generation after they were induced at 42° C. for 16 h. Primers LF and LR were designed to amplify an expected 68-bp DNA fragment from the transcripts of LFB cDNA, whereas primers PF and LR were designed to amplify an expected 380-bp DNA fragment from the contaminated plasmid DNA, which included the promoter (FIG. 5A). Results showed that three transgenic microalgae displayed a 68-bp PCR product, indicating that the LFB-DsRed fusion gene could be transcribed from the transgene SacII-cut phr-rLFB-DsRed (FIG. 5B).

### Example 6

### Protein and Western Blot Analyses

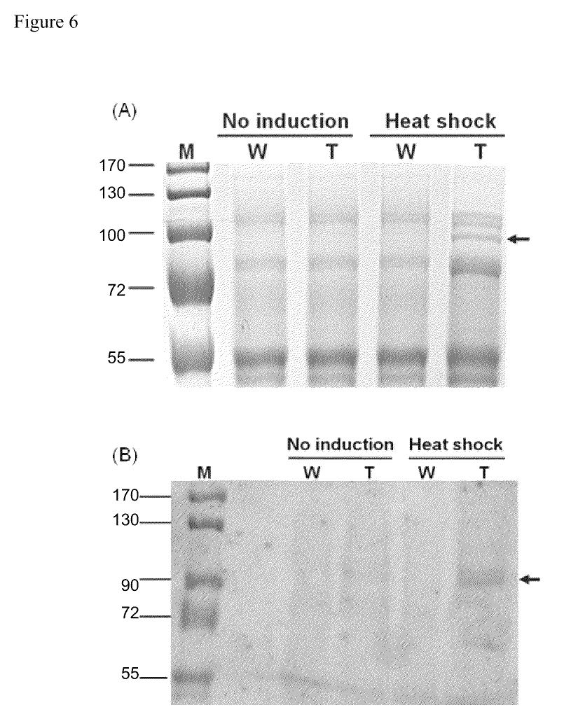

After treating the transgenic line 0829A microalga at 42° C. for 16 h, the total proteins were extracted and analyzed by SDS-polyacrylamide gel electrophoresis. Results showed that an extra peptide band with molecular mass of about 100 kDawas expressed in the transgenic microalgal line, which was absent in the proteins extracted from the wild type (FIG. 6A). This 100-kDa-protein band, which corresponded to the tetramer of fusion protein LFB-DsRed, was positive for immunological detection when analyzed by western blotting using mouse monoclonal antibody against DsRed (FIG. 6B). We also noticed that only a small amount of fusion protein was expressed before heat shock treatment (FIG. 6B), suggesting that the promoter of HSP70A combined with RBCS 2 from C. reinhardtii is not tightly controlled.

### Example 7

### Bactericidal Activity of Recombinant LFB Produced by Transgenic N. oculata

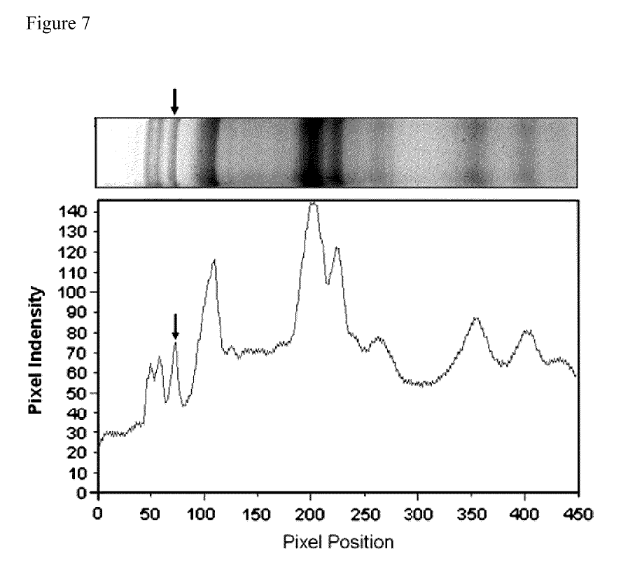

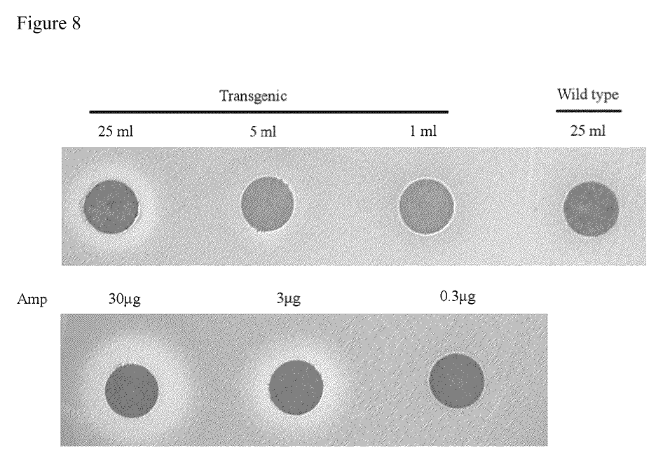

The transgenic alga 0829A was cultured at log phase, induced by heat shock, and the cells were harvested and resuspended in gastric acid for 4 h. Cell lysatewas obtained, spotted on the disc and placed on an agar plate containing V. parahaemolyticus. An inhibition zone with a diameter of 5 mm occurred if the lysate was extracted from a 25 ml culture (FIG. 7). Compared to the potency of Ampicillin on the same plate, the bactericidal efficacy of lysate from a 25 ml culture of transgenic alga line 0829A was equivalent to 8.9±0.66 mg, an average of three times that of Ampicillin, which was 0.356±0.026 mg Ampicillin per ml (or 3.56±0.26×10⁻⁸ μg Ampicillin per cell). The inhibition zone on the filter paper disc assay was also observed in the lysate extracted from 5 ml transgenic microalga, but neither in the lysate extracted from 1 ml transgenic microalga nor in the lysate extracted from 25 ml wildtype algae (FIG. 8), which indicates that bactericidal activity could be achieved from a large amount of the lactoferricin-containing transgenic microalgal line 0829A. To determine the bactericidal potency of transgenic microalga 0828A in vivo, we employed medaka fish which had been adapted from freshwater to seawater during the previous five days. Regarding the lethal condition of infection of medaka by V. parahaemolyticus, we found that the oral delivery of 1×10⁵ V. parahaemolyticus killed a medaka fish within 24 h. And, after oral delivery of 1×10⁸ transgenic microalgae per fish after 6 h enabled medaka to completely protect from the infection of 1×10⁵ of V. parahaemolyticus. To test for dose dependency, we fed medaka by the oral-in-tube method either 1×10⁶ or 1×10⁸ microalgae per fish. Six hours thereafter, V. parahaemolyticus infection was induced orally by either a high (1×10⁵ cells per fish) or a low (1×10⁴ cells per fish) dosage. Results showed that the survival rate of medaka fed with 1×10⁸ transgenic algae per fish and then infected with 1×10⁵ Vibrio cells per fish was significantly higher than that of medaka fed with the same dosage of wild-type algae and infected by same dosage of Vibrio: 85±7.1% versus 5±7.1% (Table 1). This result was also consistent with fish fed with a lower dosage (1×10⁶) of transgenic algae per fish and then infected with 1×10⁵ Vibrio cells per fish: 70% versus 5±7.1% (Table 1). The enhancement of survival of medaka after same strategy of V. parahaemolyticus infection was also concluded from the results of medaka fish infected with only one-tenth the dosage (1×10⁴ cells per fish) of V. parahaemolyticus. All of this evidence indicated the protective efficacy of transgenic microalga against Vibrio-infected fish.

TABLE 1
The survival rate of medaka fish fed with algae after V.
parahaemolyticus infection

Infection

Microalgae

Transgenic

| Injection | Dsw | Wild type | Low | High |
|---|---|---|---|---|
| Dsw | 95 ± 7.1 | 95 ± 7.1 | 95 ± 7.1 | 100 |
| Cont | 90 | 100 | 100 | 95 ± 7.1 |
| VP-L | 15 ± 7.1 | 10 | 65 ± 21.2 | 95 ± 7.1 |
| VP-H | 0 | 5 ± 7.1 | 70 | 85 ± 7.1 |

Medaka fish were fed orally-in-tube with microalga N. oculata, and 6 h later were orally-in-tube injected V. parahaemolyticus. The survival rate of medaka was calculated 24 h after V. parahaemolyticus infection on the basis of examining 10 medaka fish. Dsw: 10 μl distilled seawater; Cont: high dosage of dead (boiled for 30 min) V. parahaemolyticus (1×10⁵ cells per fish) which served as negative control; VP-L: low dosage of living V. parahaemolyticus (1×10⁴ cells per fish); VP-H: high dosage of living V. parahaemolyticus (1×10⁵ cells per fish); wild type: wild-type N. oculata (1×10⁸ cells per fish); TL: low dosage of transgenic N. oculata (1×10⁶ cells per fish); and TH: high dosage of transgenic N. oculata (33 10⁸ cells per fish). The data were presented by the average of two independent trials.

### Example 8

### Biological activity of VP28 Produced by Transgenic N. oculata

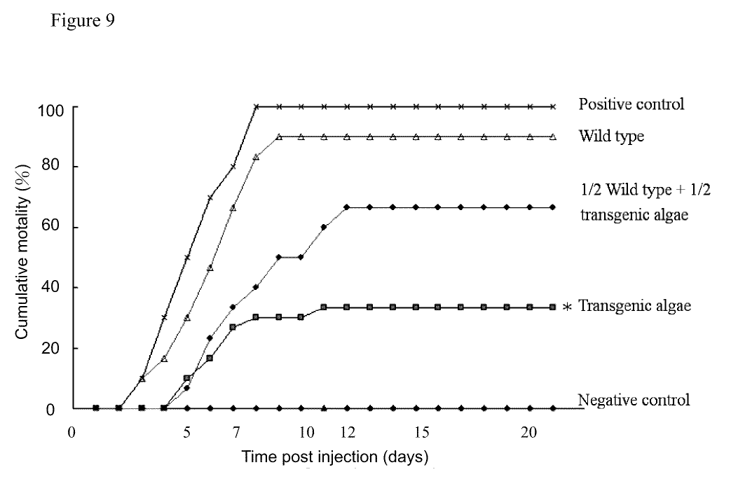

Penaeus monodon was injected with WSSV to perform the oral vaccine infection test. The mortality of Penaeus monodon was calculated on specific days. The Statistic results of cumulative mortality of Penaeus monodon were from each experimental group with different feed, such as wild-type algae, half wild-type algae plus half transgenic algae, transgenic algae, and general feed (positive and negative control) (N=10). Groups of positive and negative control, transgenic algae, wild-type and half wild-type algae plus half VP28 transgenic algae were injected with WSSV solution. Group of negative control were injected with PBS. The data were presented by the average of three replications (FIG. 9).

### Example 9

### Biological Activity of rYGH Produced by Transgenic N. oculata

Tilapia nilotica were fed with wild-type algae or transgenic algae comprising rYGH gene of Acanthopagrus latus through Artemia saline, and their grow rates were recorded. The results showed that Tilapia nilotica fed with transgenic algae comprising rYGH gene of Acanthopagrus latus grown rapidly, with weight gain rate 2 to 3 times and body length almost 2 times than the group fed with wild-type algae (Table 2). This evidence indicated the fish growth factor produced by transgenic algae had biological activity.

TABLE 2
Comparison of growth rate of Tilapia nilotica fed with wild-type algae
or transgenic algae comprising rYGH gene of Acanthopagrus latus
through Artemia saline.

| | | | | | | | Body | |
|---|---|---|---|---|---|---|---|---|
| Culture | | | | Weight | Average Body Length | | Length | |
| Time | | Average Weight (g) | | Gain | (cm) | | Increased | Condition |
| (hour) | Feed | Initial | Final | (%) | Initial | Final | (%) | Factor |
| 4 | Wild-type | 0.23 ± 0.01^{a} | 0.43 ± 0.07^{a} | 87 ± 3.2^{a} | 1.36 ± 0.01^{a} | 3.27 ± 0.10^{a} | 139 ± 4.2^{a} | 12.0 ± 0.01^{a} |
| | Transgenic | 0.23 ± 0.01^{a} | 0.77 ± 0.09^{b} | 232 ± 7.1^{b} | 1.36 ± 0.01^{a} | 3.98 ± 0.17^{b} | 197 ± 7.5^{b} | 12.1 ± 0.02^{a} |
| 6 | Wild-type | 0.23 ± 0.01^{a} | 0.47 ± 0.06^{c} | 104 ± 4.1^{c} | 1.36 ± 0.01^{a} | 3.35 ± 0.13^{c} | 146 ± 5.5^{c} | 12.2 ± 0.02^{a} |
| | Transgenic | 0.23 ± 0.01^{a} | 0.98 ± 0.14^{d} | 316 ± 9.7^{d} | 1.36 ± 0.01^{a} | 4.38 ± 0.23^{d} | 217 ± 8.9^{d} | 12.0 ± 0.01^{a} |

The data were presented by the average value ± SD.
n = 20 (Tilapia nilotica fry) in each group.
The average values of the same column marked the same letters meant no significant difference (p < 0.05).
100 mL N. oculata (1 × 10⁵ cells) were used.

One skilled in the art readily appreciates that the present invention is well adapted to carry out the objects and obtain the ends and advantages mentioned, as well as those inherent therein. The plants, animals, and processes and methods for producing them are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations, which are not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A modified nucleic acid for expressing bovine lactoferricin (LFB) in algae, which consists of SEQ ID NO: 3.

2. The nucleic acid of claim 1, which is further operably linked to a sequence encoding fluorescent protein and is translated to a fusion protein of LFB and fluorescent protein.

3. The nucleic acid of claim 2, wherein the fluorescent protein is red fluorescent protein (DsRed), providing a sequence consisting of SEQ ID NO: 2 which is translated to a fusion protein of LFB and red fluorescent protein (LFB-DsRed).

4. The nucleic acid of claim 3, which comprises a pepsin cutting site located in the junction between LFB and DsRed.

5. The nucleic acid of claim 2, wherein the fluorescent protein is a fluorescent marker for screening transgenic algae.

6. The nucleic acid of claim 2, which is further operably linked to a promoter, wherein the promoter consists of SEQ ID NO: 1.

7. The nucleic acid of claim 1, wherein the algae is microalgae.

8. A method of producing a foreign desired gene product in algae, comprising:
(a) weakening or removing cell wall of the algae by a protein enzyme solution to make algae become suitable for gene transfer;
(b) transferring a foreign gene encoding the desired gene product into the algae; and
(c) expressing the foreign gene to produce the desired gene product.

9. The method of claim 8, wherein the protein enzyme solution comprises a protein enzyme and a solution providing an environment in which said enzyme is activated.

10. The method of claim 8, wherein the foreign gene comprises a gene encoding a fusion protein of LFB and red fluorescent protein (LFB-DsRed), a gene encoding an envelop protein VP28 of white spot syndrome virus, or a gene encoding growth factor rYGH of Acanthopagrus latus.

11. The method of claim 8, wherein the transferring of the foreign gene encoding the desired gene product into the algae is accomplished by electroporation.

12. The method of claim 8, wherein the desired gene product expressed in algae has biological activity.

13. The method of claim 12, wherein the biological activity comprises antibacterial activity, antiviral activity, or increasing growth rate.

14. The method of claim 13, wherein the biological activity comprises antibacterial activity for Vibrio parahaemolyticus, antiviral activity for white spot syndrome virus, or increasing growth rate 2 to 3 times.

15. A feed composition comprising a transgenic algae or its offspring, wherein the cell wall of said transgenic algae is weakened or removed by a protein enzyme solution.

16. The composition of claim 15, wherein the protein enzyme solution comprises a protein enzyme and a solution providing an environment in which said enzyme is activated.

17. The composition of claim 15, wherein the transgenic algae comprises a foreign gene encoding a fusion protein of LFB and red fluorescent protein (LFB-DsRed), a foreign gene encoding an envelop protein VP28 of white spot syndrome virus, or a foreign gene encoding growth factor rYGH of Acanthopagrus latus.

18. The composition of claim 17, wherein the foreign gene is expressed in algae and produce a desired gene product which comprises biological activity of antibacterial activity, antiviral activity, or increasing growth rate.

19. The composition of claim 15, which is used to feed an aquatic organism.

20. The composition of claim 19, wherein the aquatic organism is fish or shrimp.


SEQUENCE LISTING (16 sequences)

SEQ ID NO: 1 - DNA, 520 bp, Artificial
  A promoter of heat shock protein 70A gene fused with a promoterof ribose-1,5-bisphosphate carboxylase/oxygenase small subunit 2gene of Chlamydomonase reinhardtii.
cggcggggagctcgctgaggcttgacatgattggtgcgtatgtttgtatgaagctacaggactgatttggcgggctatgagggcgcgggaagctctggaagggccgcgatggggcgcgcggcgtccagaaggcgccatacggcccgctggcggcacccatccggtataaaagcccgcgaccccgaacggtgacctccactttcagcgacaaacgagcacttatacatacgcgactattctgccgctatacataaccactcagctagcttaagatcccatcaagcttgcatgccgggcgcgccagaaggagcgcagccaaaccaggatgatgtttgatggggtatttgagcacttgcaacccttatccggaagccccctggcccacaaaggctaggcgccaatgcaagcagttcgcatgcagcccctggagcggtgccctcctgataaaccggccagggggcctatgttctttacttttttacaagagaagtcactcaacatcttaaaatggccaggtgag

SEQ ID NO: 2 - DNA, 775 bp, Artificial
  Recombinant algae-codon-optimized LFB plus red fluorescent protein gene of Discosoma sp (Clontech.)
gctagcaccggtcgccaccatgttcaaatgtcgtcgttggcaatggcgtatgaaaaaattaggtgctccttctattacatgtgtacgtcgtgctttcatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgtag

SEQ ID NO: 3 - DNA, 78 bp, Artificial
  An algae-codon-optimized LFB cDNA
atgttcaaatgtcgtcgttggcaatggcgtatgaaaaaattaggtgctccttctattacatgtgtacgtcgtgctttc

SEQ ID NO: 4 - DNA, 49 bp, Artificial
  PCR forward primer
gctagcaccggtcgccaccatgttcaaatgtcgtcgttggcaatggcgt

SEQ ID NO: 5 - DNA, 39 bp, Artificial
  PCR forward primer
gcaatggcgtatgaaaaaattaggtgctccttctattac

SEQ ID NO: 6 - DNA, 39 bp, Artificial
  PCR forward primer
cttctattacatgtgtacgtcgtgctttcatggcctcct

SEQ ID NO: 7 - DNA, 33 bp, Artificial
  PCR reverse primer
atttgtgatgctattgctttatttgtaaccatt

SEQ ID NO: 8 - DNA, 615 bp, white spot syndrome baculovirus
  gene (1)..(615)
atggatctttctttcactctttcggtcgtgtcggccatcctcgccatcactgctgtgattgctgtatttattgtgatttttaggtatcacaacactgtgaccaagaccatcgaaacccacacagacaatatcgagacaaacatggatgaaaacctccgcattcctgtgactgctgaggttggatcaggctacttcaagatgactgatgtgtcctttgacagcgacaccttgggcaaaatcaagatccgcaatggaaagtctgatgcacagatgaaggaagaagatgcggatcttgtcatcactcccgtggagggccgagcactcgaagtgactgtggggcagaatctcacctttgagggaacattcaaggtgtggaacaacacatcaagaaagatcaacatcactggtatgcagatggtgccaaagattaacccatcaaaggcctttgtcggtagctccaacacctcctccttcacccccgtctctattgatgaggatgaagttggcacctttgtgtgtggtaccacctttggcgcaccaattgcagctaccgccggtggaaatcttttcgacatgtacgtgcacgtcacctactctggcactgagaccgagtaa

SEQ ID NO: 9 - DNA, 615 bp, Acanthopagrus latus
  gene (1)..(615)
atggacagagtggtgctcatgctgtcggtgctgtctctgggcgtctcctctcagccgatcacagacggccagcgtctgttctccatcgctgtcagcagagttcaacacctccacctgctcgctcagagactcttctctgactttgagagctctctgcagactgaggagcaacgacagctcaacaaaatcttcctgcaggatttctgtaactctgattacatcatcagccccatcgacaagcacgagacacagcgcagctcagtgttgaagctgctgtctatctcctatcgattggtcgagtcttgggagttccccagtcgttctctggctggcggttctgctccaaggaaccagatttcacccaaactgtctgagctgaagacaggcatccatctcctgatcagggccaatgaggatggagcagagctcttccctgatagctccgccctccagctggctccttatggagactactaccaaagtccgggcaccgacgagtcgctgagacgaacctacgaactacttgcctgtttcaaaaaagacatgcacaaggtggagacctacctgacagtggcaaaatgtagactctctccagaggccaactgcaccctgtag

SEQ ID NO: 10 - DNA, 26 bp, Artificial
  Specific primers synthesized for detetion of the existence of the transferred DNA fragment.
cctcctccgagaacgtcatcaccgag

SEQ ID NO: 11 - DNA, 21 bp, Artificial
  Specific primers synthesized for detetion of the existence of the transferred DNA fragment.
cctcggtgcgctcgtactgct

SEQ ID NO: 12 - DNA, 26 bp, Artificial
  Primers for detection of the endogenous 18S rRNA gene.
gcggaggaaaagaactaaccaggatt

SEQ ID NO: 13 - DNA, 19 bp, Artificial
  Primers for detection of the endogenous 18S rRNA gene.
aacgccatggcacaaccgc

SEQ ID NO: 14 - DNA, 21 bp, Artificial
  Primer used to amplify an expected 68-bp PCR product from the transcript of LFB cDNA.
gtcgttggcaatggcgtatga

SEQ ID NO: 15 - DNA, 26 bp, Artificial
  Primer used to amplify an expected 68-bp PCR product from the transcript of LFB cDNA, also used to amplify a 380-bp PCR fragment from the contaminated plasmid DNA which is not eliminated by DNase.
gccatgaaagcacgtacacatgtaat

SEQ ID NO: 16 - DNA, 24 bp, Artificial
  Primer used to amplify a 380-bp PCR fragment from the contaminated plasmid DNA which is not eliminated by DNase.
cgctgaggcttgacatgattggtg